(19) United States
(12) Patent Application Publication
(10) Pub. No.: US 2026/0232806 A1
(43) Pub. Date: Aug. 13, 2026

(54) Compositions and Methods for Treating Wound Healing and Hypothermia

(71) Applicant: Board of Regents, The University of Texas System, Austin, TX (US)

(72) Inventors: Kreg Zimmern, San Antonio, TX (US); Maria Gonzalez Porras, San Antonio, TX (US); Eric Brey, San Antonio, TX (US); Achraf Noureddine, Albuquerque, NM (US)

(21) Appl. No.: 19/536,074

(22) Filed: Feb. 10, 2026

Related U.S. Application Data
(60) Provisional application No. 63/756,780, filed on Feb. 10, 2025.

(51) Int. Cl. A61K 45/06; A61K 9/00; A61K 9/51; A61K 38/00

(52) U.S. Cl. CPC A61K 45/06; A61K 9/0014; A61K 9/0019; A61K 9/5161; A61K 38/00

(57) ABSTRACT

Aspects describe nanoparticle-delivered adenyl cyclase activators (e.g., forskolin via LCMSN) to induce sWAT browning, enhancing thermogenesis for wound healing and cold injury protection. Applications include chronic wounds, diabetic ulcers, burns, frostbite, and hypothermia via subcutaneous or transdermal routes, with demonstrated acceleration of closure, reduced inflammation/necrosis, and improved angiogenesis/ECM remodeling in frostbite models.

### CROSS-REFERENCE TO RELATED APPLICATIONS

This application claims the benefit under 35 U.S.C. § 119(e) of U.S. Provisional Application No. 63/756,780, filed Feb. 10, 2025, which is incorporated herein by reference in its entirety.

### STATEMENT REGARDING FEDERALLY FUNDED RESEARCH

None.

### FIELD OF INVENTION

Certain aspects are directed to the field of medicine, in particular wound healing, cold-induced injuries, and thermogenic adipose tissue modulation.

### BACKGROUND

Impaired wound healing affects millions in the U.S. each year, costing around $20billion (15). Various factors can disrupt the healing stages, including prolonged inflammation, excessive mechanical tension, and abnormal matrix degradation (16-18). Treatment options focus on supporting keratinocytes, fibroblasts/myofibroblasts, and macrophages, using methods like dressings, negative-pressure therapy, lasers, corticosteroids, and growth factors (15, 19, 20). Recent advances in regenerative medicine have sought to harness the body's own biological processes to accelerate wound healing, including the use of bioactive molecules, engineered nanoparticles, and cell-based therapies. Clinicians increasingly use fat autografts to minimize scarring and enhance healing (21-27).

Infections remain a concern, especially in complex wounds or compromised hygiene settings, with rising antibiotic resistance complicating treatment. Diabetic ulcers and pressure sores resist healing due to underlying conditions. Scar formation trades off with effective healing, and advanced treatments like grafts or regenerative medicine are not universally accessible. Pain during frequent dressing changes hinders care. Poor circulation, diabetes, or immune deficiencies prolong healing.

For frostbite, rapid rewarming risks reperfusion injury and further damage. Thrombolytics help severe cases but carry bleeding risks. Frostbite often occurs in remote/extreme conditions (e.g., military operations, mountaineering), delaying care and exacerbating damage. Long-term issues include chronic pain, cold sensitivity, and permanent tissue loss. Frostbite research is underfunded compared to other fields.

Recent studies show subcutaneous white adipose tissue (sWAT) undergoes browning (to beige adipose tissue, bAT) after skin injury, supporting repair via thermogenesis, immune modulation (e.g., M1-to-M2 macrophage shift), and angiogenesis [Cai et al., 2024]. In larger wounds, sWAT is the primary adipocyte source. Beige adipocytes secrete factors regulating macrophages and fibroblasts. However, mechanisms linking thermogenic activation to improved outcomes in chronic wounds or cold injuries remain underexplored.

There remains a need for compositions and methods enhancing thermogenic adipose tissue to treat wounds, frostbite, hypothermia, and related conditions, particularly via targeted/local delivery and non-invasive routes.

### SUMMARY

This application describes a solution to the problems associated with treating wounds and frostbite by using compositions that transitions white adipose tissue (WAT) to beige adipose tissue (bAT).

This application describes compositions and methods using adenyl cyclase activators (e.g., forskolin) delivered via nanoparticles (e.g., lipid-coated mesoporous silica nanoparticles, LCMSN) to induce browning of sWAT to beige adipose tissue (bAT), promoting thermogenesis, anti-inflammatory effects, angiogenesis, and tissue repair. Aspects target chronic wounds (diabetic ulcers, burns, lacerations), frostbite, cold-related injuries, and hypothermia by maintaining local/core temperature and supporting healing. Embodiments include subcutaneous injection or transdermal delivery (e.g., creams, dissolvable microneedles) for pharmacological agents like forskolin. Nanoparticles enable sustained local release, overcoming forskolin's poor bioavailability and retention. Additional nanoparticle embodiments include matrix-based particles (e.g., solid lipid nanoparticles or polymeric PLGA particles) prepared by high-pressure homogenization/solvent extraction, liposomal vesicles formed by thin-film rehydration or homogenization, and micellar systems for hydrophobic partitioning, all capable of delivering the adenyl cyclase activator to induce browning and thermogenesis. Increasing bAT enhances wound healing by promoting M2 macrophage polarization, VEGF/CD31 expression for angiogenesis, ECM remodeling, and reduced inflammation. In frostbite models, LCMSN-forskolin accelerates closure, reduces necrosis/fibrosis, modulates immune markers, and improves histopathological outcomes. Certain aspects can be used to treat or prevent hypothermia or cold-related injuries by maintaining local and core temperature. In battlefield-relevant scenarios, such as combined tourniquet application and distal frostbite injury in extremities, the compositions and methods described herein are particularly advantageous for reducing severity of ischemia/reperfusion-exacerbated cold damage and supporting healing under conditions of restricted perfusion.

Certain aspects are directed to increasing subcutaneous bAT in wound healing and providing a novel therapeutic approach for enhancing tissue repair, particularly in challenging conditions such as frostbite, diabetic ulcers, burns, and other chronic wounds. By leveraging the ability of adipose tissue to transition into a thermogenic and metabolically active state, a faster and more efficient wound healing process is promoted to improve patient outcomes. Described herein are methods and compositions for increasing beige adipose tissue by using nanoparticles and/or transdermal delivery mechanisms for pharmacological agents such as adenyl cyclase activators, e.g., forskolin. Certain aspects can be used to treat or prevent hypothermia or cold-related injuries by maintaining local and core temperature.

Adenyl cyclase activators are compounds or molecules that enhance the activity of adenyl cyclase, an enzyme crucial for converting ATP to cyclic AMP (cAMP). cAMP is a key secondary messenger in many biological processes, including signal transduction pathways that regulate cell growth, differentiation, and metabolism.

Adenyl cyclase activators work by directly or indirectly increasing the production of cAMP. This can be achieved by (i) stimulating G-protein coupled receptors (GPCRs) that are linked to stimulatory G proteins (Gs), which activate adenyl cyclase; (ii) directly binding to and activating adenyl cyclase; or (iii) inhibiting phosphodiesterases, which break down cAMP, thereby indirectly increasing cAMP levels.

Examples of Adenyl Cyclase Activators include but are not limited to forskolin (also known as coleonol, IUPAC name (3R,4aR,5S,6S,6aS,10S,10aR,10bS)-3,4a,5,6,6a,10,10a,10b-octahydro-3,5,6-trihydroxy-6a,10a-dimethyl-4H- 1-benzofuro[3,2-c]pyran-4-one), GTPgS and its analogs, β-Adrenergic Agonists (e.g., isoproterenol), prostaglandin E1 (PGE1), calcium ionophores (e.g., A23187), diterpenes like 1,9-dideoxyforskolin, and compounds with similar activities.

Certain embodiments are directed to a method for treating a wound or cold-damaged tissue comprising administering to the wound or the cold-damaged tissue a nanoparticle comprising an adenyl cyclase activator. The nanoparticle can be a lipid coated mesoporous silica nanoparticle. In certain aspects the adenyl cyclase activator is forskolin. The nanoparticle composition can be administered by subcutaneous injection or topical gel or substrate. The wound can be a chronic wound. In certain aspects the wound is frostbite, a diabetic ulcer, a laceration, or a burn. In certain aspects the nanoparticle composition can comprise a second therapeutic agent, e.g., an antibiotic or other agent.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The term “beige adipose tissue” refers to a type of fat cell that combines characteristics of both white adipose tissue (WAT) and brown adipose tissue (BAT). Beige fat cells are morphologically and functionally between white and brown adipocytes. They have the capacity to burn calories through thermogenesis (heat production), similar to brown fat, but they are found interspersed within white fat depots. Like brown fat, beige adipocytes contain more mitochondria than white fat cells, and these mitochondria are equipped with uncoupling protein 1 (UCP1). UCP1 uncouples oxidative phosphorylation from ATP synthesis, allowing energy from food to be released as heat instead of being stored as fat, which is particularly important for non-shivering thermogenesis. One of the most notable features of beige fat is its plasticity. White fat cells can be induced to take on the characteristics of beige fat through various stimuli like cold exposure, certain hormones, or particular drugs. This process is called “browning” of white adipose tissue.

The term “thermogenic” refers to the process by which organisms produce heat, which is crucial for maintaining body temperature, especially in endothermic (warm-blooded) animals like mammals and birds. Shivering thermogenesis involves muscle activity to generate heat by shivering. Non-shivering thermogenesis relies on brown or beige adipose tissues, which contain numerous mitochondria with uncoupling protein 1 (UCP1), which dissipates energy as heat rather than using it for ATP synthesis. Cold exposure is a natural thermogenic stimulus that activates brown and beige fat to produce heat.

The term “chronic wound” refers to a wound that does not progress through the normal stages of healing within an expected or reasonable timeframe. Typically, a wound is considered chronic if it does not heal within 3 months despite standard treatment. Normal wound healing involves stages like hemostasis, inflammation, proliferation, and remodeling. Chronic wounds fail to progress through these phases appropriately, often getting stuck in one phase.

The term “frostbite” refers to a medical condition where skin and underlying tissues freeze due to exposure to extreme cold. Frostbite occurs when the body's natural mechanisms for maintaining warmth fail in extremely cold conditions. The blood vessels near the skin constrict to preserve core body temperature, reducing blood flow to extremities, which can lead to ice crystal formation within the cells. Frostbite includes cold-induced tissue damage with ice crystal formation, ischemia, and reperfusion injury.

The term “wound healing” refers to a complex and dynamic process by which the body repairs or replaces damaged tissue. It involves multiple stages: hemostasis, inflammation, proliferation and remodeling.

The term “subcutaneous” refers to the layer of tissue directly beneath the skin, specifically under the dermis. This layer is composed mainly of fat (adipose tissue), connective tissue, blood vessels, and nerves. It serves as an insulating layer, helping to regulate body temperature, provide energy storage, and act as a cushion for the body. Many medications or vaccines are administered subcutaneously because absorption rates from this layer are generally slower than intramuscular injections but faster than intradermal ones. Common sites for subcutaneous injections include the abdomen, thighs, upper arms, or the back of the upper arm. Subcutaneous tissue can be involved in surgical procedures, both as a layer to be navigated through to reach deeper structures or as part of the healing process where it might be affected by scarring or infection.

The term “mesoporous silica” refers to a form of silica (SiO?) characterized by its mesoporous structure, which means it has pores with diameters in the range of 2 to 50 nanometers (nm). Mesoporous silica features ordered arrays of uniform pores, often arranged in patterns like hexagonal, cubic, or lamellar structures. This ordered porosity distinguishes it from amorphous or non-porous silica.

The term “reperfusion” refers to the restoration of blood flow to an organ or tissue that has experienced a period of ischemia (lack of blood supply due to obstruction or constriction of blood vessels). This process is critical in many medical scenarios, particularly after events like myocardial infarction (heart attack), stroke, or limb ischemia. When blood flow is restored, oxygen and nutrients are once again supplied to the deprived area, oxygenated blood clears metabolic waste, and cells can resume their normal functions.

The term “administering” refers to giving therapeutics to patients through various routes like oral, intravenous (IV), intramuscular (IM), subcutaneous, or topical application.

The term “nanoparticle” refers to a particle with at least one dimension sized from 1 to 100 nanometers (nm). This size scale gives nanoparticles unique physical, chemical, and biological properties compared to their bulk material counterparts due to quantum effects and increased surface area to volume ratio.

The term “injection” refers to the act of introducing a substance, such as a liquid medication, drug, or other fluid, into the body via a needle or syringe. Sites of administration can include intravenous, intramuscular, subcutaneous, intradermal, intraosseous, and intraperitoneal.

The term “forskolin” refers to a chemical compound found in the roots of the Indian coleus plant (Coleus forskohlii). Forskolin is a diterpene, specifically classified as a labdane diterpene, known for its unique cyclic structure which includes a furan ring. Forskolin directly activates adenylate cyclase, an enzyme that catalyzes the conversion of ATP to cyclic AMP (cAMP). This increase in intracellular cAMP levels can affect numerous cellular processes because cAMP acts as a secondary messenger in many signal transduction pathways. By elevating cAMP, forskolin influences pathways related to vasodilation, bronchodilation, lipolysis, inhibition of platelet aggregation, and hormone secretion.

The term “diabetic ulcer” refers to an open sore or wound, most commonly occurring on the feet, but can also develop on other parts of the lower extremities. These ulcers are more frequent in individuals with diabetes due to the complex interplay of factors related to the disease. They are specifically associated with diabetic neuropathy (nerve damage) and peripheral artery disease (poor circulation).

The term “laceration” refers to a type of wound where the skin or other tissues are torn, cut, or irregularly ripped, often as a result of blunt trauma, sharp objects, or significant force. Lacerations typically have jagged or irregular edges, distinguishing them from the cleaner, straight cuts associated with incisions. They can be superficial, involving only the skin, or deep, affecting underlying structures like muscles, tendons, nerves, or blood vessels.

The term “burn” refers to an injury to the skin or other tissues caused by heat, radiation, electricity, chemicals, or friction. They can be classified by first to fourth degree depending on the depth of the burn.

The terms “treat”, “treating” and “treatment” are used to refer to any action providing a benefit to a patient at risk for or afflicted with a disease, including improvement in the condition through lessening, inhibiting, suppressing, or eliminating at least one symptom, delaying progression of the disease, delaying or inhibiting the likelihood of the onset of the disease, etc.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Overview of the mechanisms of increasing thermogenic adipose tissue to enhance wound healing.

FIG. 2A-2F. LCMSN loaded with forskolin (LCMSN-F) can increase thermogenic activity in adipocytes in vitro and in vivo. (A) TEM images of implemented MSN. Scale bar=200 nm. (B) 3D confocal microscopy image showing LCMSN uptake in mature adipocytes. (C) Relative mRNA expression of UCP1 in adipocytes after treatment with LCMSN-F. (D) In vivo image of mice after 48 h post subcutaneous injection. Relative (E) mRNA and (F) protein expression of UCP1 in inguinal AT after subcutaneous injection with LCMSN-F, F or CTL.

FIG. 3A-3C. (A) Arg1 gene expression in macrophages in vitro after 48 h exposure to white (WAT) and beige (bAT) adipocyte conditioned media (CM). (B-C) Arg1 expression in sWAT after 6h or 48 h LCMSN-FSK subcutaneous injection. Angiogenic markers gene expression in adipose tissue after LCMSN-FSK subcutaneous injection.

FIG. 4A-4B. Angiogenic markers gene expression in adipose tissue after LCMSN-FSK subcutaneous injection, (A) CD31 and (B) VEGF.

FIG. 5 Proposed strategy for research. Nanoparticle-mediated forskolin delivery increases local temperature preventing and/or treating cold-related injuries.

FIG. 6A-6B. (A) Thermal images of whole mice captured using a FLUKE TiS45 infrared camera of mice undergoing a cold challenge after being injected with either protocells-FSK or saline (CTL). The images show that mice injected with LCMSN-FSK maintained a higher surface temperature compared to the control (CTL) group. (B) Rectal temperature measurements confirmed that protocells-FSK-treated mice better maintained their core temperature compared to CTL mice.

FIG. 7A-7G. Experimental Design and temporal characterization of frostbite wound healing in dermal and subcutaneous layers using a mouse model. (A) Schematic of dorsal frostbite injury model. (B) Representative H&E images of normal skin and frostbite wound on Day 7, 14, and 21, which shows progressive re-epithelialization and wound healing over time. Time course RT-qPCR analysis of (C) thermogenic/adipocyte markers (Ucp1, Adiponectin), (D) angiogenesis/endothelial markers (Vegfα, Cd31), (E) pro-inflammatory cytokines (Il1b, Tnfα), (F) inflammation resolution markers (Il10, Arg1), and (G) extracellular matrix remodeling/fibrosis markers (Col1a1, Col3a1) in dermal and subcutaneous adipose tissue layers harvested on D4, 7, 14, and 21. Gene expression are normalized to no wound control samples. Bars represent mean±SEM; statistical significance is indicated as shown in the plots (*p<0.05,**p<0.01, ***p<0.001, ****p<0.0001).

FIG. 8A-8E. Lipid coated mesoporous silica nanoparticles loaded with forskolin treatment accelerates frostbite wound closure and improves histopathological wound characteristics. (A) Representative wound images from PBS and LCMSN-FSK treated mice on Day 4, 7, 14, and 21 following frostbite injury. (B) Quantification of wound size closure (%) normalized to day 4 wound area, demonstrating greater reduction in wound area over time with LCMSN-FSK treatment compared with PBS. Semi-quantitative histopathology scoring of wound sections at D7, 14, and 21 for (C) epidermal loss, (D) necrosis, and (E) fibrosis, showing improved epidermal recovery and reduced necrotic burden with LCMSN-FSK during the mid wound healing timepoint. Fibrosis peaked during the remodeling phase and trended lower with LCMSN-FSK at later time points. Data are presented as mean±SEM with individual biological replicates; statistical significance is indicated as shown (*p<0.05).

FIG. 9A-9B. LCMSN-FSK modulates adipose function, extracellular matrix remodeling, and angiogenic associated gene expressions in dermal and subcutaneous tissue layers after cold induced injury. (A) Representative Masson's trichrome staining of normal skin and frostbite wound from PBS and LCMSN-FSK treated mice on Day 7, 14, and 21, Collagen rich regions were stained blue, highlighting progressive ECM remodeling during healing; arrows indicate healthy adipocytes. (B) RT-qPCR analysis of wound-healing markers in dermal and subcutaneous fat tissue layers harvested on Day 4, 7, 14, and 21 from PBS and LCMSN-FSK treated wounds. Expression is presented as fold change relative to no wound control samples. Gene panel include adipose/thermogenic markers (Ucp1, Adiponectin), vascular associated markers (Vegfα, Cd31), and extracellular matrix remodeling markers (Col1a1, Col3a1). Bars represent mean±SEM with individual biological replicates shown; statistical significance is indicated as shown (*p<0.05, **p<0.01, ***p<0.001, ****p<0.0001).

FIG. 10A-10B. LCMSN-FSK modulates inflammatory and anti-inflammatory immune gene marker expression during cold induced wound healing in dermal and subcutaneous tissues. (A) RT-qPCR analysis of immune responsive wound healing markers in dermal and subcutaneous fat tissue layers harvested on Day 4, 7, 14, and 21 from PBS and LCMSN-FSK treated wounds. Expression is presented as fold change relative to no wound control samples. Overall, LCMSN-FSK was shown to reduce the pro-inflammatory (M1-associated) cytokines (Il1b, Tnfα) expression and enhances or shifts the temporal expression of anti-inflammatory (M2-associated) markers (Il10, Arg1) during wound healing. (B) Quantitative histopathology scoring of overall inflammation in wound tissue on Day 7, 14, and 21 comparing PBS and LCMSN-FSK treatment groups. Bars represent mean±SEM with individual biological replicates shown; statistical significance is indicated as shown (*p<0.05, **p<0.01, ***p <0.001, ****p<0.0001).

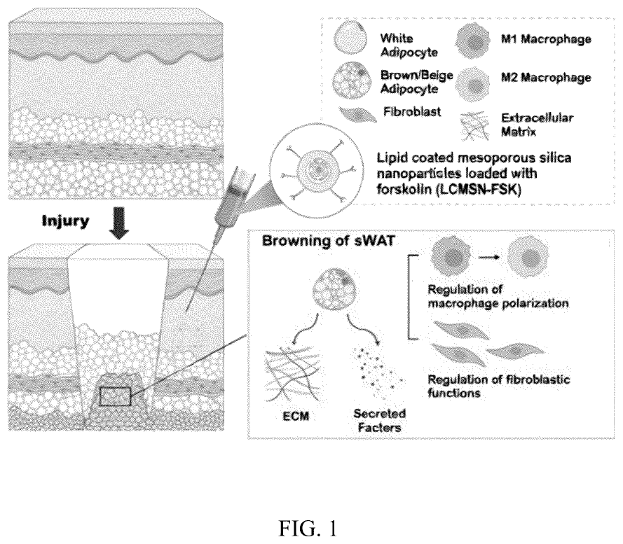

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

### I. Thermogenic (Beige) Fat as a Mechanism for Wound Healing and Hypothermia

Wound healing is a complex and dynamic process involving multiple stages, including hemostasis, inflammation, proliferation, and remodeling (3). Effective wound repair relies on a finely tuned balance of cellular and molecular events, including immune cell infiltration, angiogenesis, extracellular matrix (ECM) remodeling, and tissue regeneration (5). Failure or delay in any of these stages can lead to chronic wounds, impaired healing, or excessive scarring. Significant injuries often require several weeks to heal and typically result in fibrotic scarring, which can impair tissue function. Developing effective therapeutics to prevent scarring and promote the repair of chronic wounds needs a deeper understanding of the cellular and molecular mechanisms underlying wound healing.

Adipocytes actively migrate to epidermal wounds and contribute to tissue repair (6). In small wounds, dermis-derived adipocytes are primarily involved (7). However, in larger wounds, subcutaneous white adipose tissue (sWAT) becomes the main source of adipocytes in wound repair (8, 9). Moreover, sWAT undergoes browning—its transformation into beige or brown-like adipose tissue (bAT)—after skin injury (9). There is great potential in taking advantage of the plasticity of adipocytes and manipulating them in wound healing. Methods and compositions have been developed to increase thermogenic activity in sWAT, these methods and composition can be employed for use in wound healing.

In military and austere environments, frostbite frequently occurs concurrently with extremity ischemia due to tourniquet application for hemorrhage control. Reduced blood flow accelerates cooling of distal tissue, and reperfusion upon tourniquet release introduces additional oxidative injury. The present compositions and methods, by locally inducing thermogenic activity in subcutaneous adipose tissue, raise tissue temperature, mitigate ischemic cooling, attenuate reperfusion damage, and promote resolution of inflammation and angiogenesis—offering a novel early-intervention strategy for combined tourniquet-frostbite injuries in combat casualties. Preliminary models combining 2-hour tourniquet application with distal cold injury in rats demonstrate significant muscle edema, hemorrhage, and histological damage, underscoring the need for therapies that address both thermal and ischemic components of injury.

Adipocytes serve as the primary energy storage depot in the body and have essential endocrine roles (10). They are located both peripherally and within various tissues, where they significantly contribute to the regulation of tissue repair (11,12). These cells are highly dynamic, capable of enlarging to store fat and shrinking through lipid loss or lipolysis (13). Different stimuli can trigger adipocytes to undergo transitions that allow them to adopt specialized functions, such as thermogenesis and contractility. Their plasticity enables them to influence tissue repair and fibrosis in organs like the skin, heart, skeletal muscle, and mammary gland by releasing bioactive substances, lipids, and adipokines, as well as by transdifferentiating into contractile fibroblasts (14). After injury, sWAT rises to the wound bed during the early stages of healing, helping to close the wound with some adipocytes transforming into fibroblasts to aid in tissue repair (9).

Local stimulation of thermogenic activity in sWAT, by inducing sWAT transition to beige adipose tissue, will enhance wound healing. WAT and BAT differ in their secretory profiles and in their ECM composition, potentially influencing various cells within the wound healing microenvironment. These differences could impact critical processes, including immune regulation and tissue repair. Various factors can disrupt the healing stages, including prolonged inflammation, excessive mechanical tension, and abnormal matrix degradation (16-18). Treatment options focus on supporting keratinocytes, fibroblasts/myofibroblasts, and macrophages, using methods like dressings, negative-pressure therapy, lasers, corticosteroids, and growth factors (15, 19, 20). Recent advances in regenerative medicine have sought to harness the body's own biological processes to accelerate wound healing, including the use of bioactive molecules, engineered nanoparticles, and cell-based therapies. Clinicians increasingly use fat autografts to minimize scarring and enhance healing (21-27).

Increasing subcutaneous beige adipose tissue (bAT) in wound healing provides a novel therapeutic approach for enhancing tissue repair, particularly in challenging conditions such as frostbite, diabetic ulcers, burns, and other chronic wounds. By leveraging the ability of sWAT to transition into a thermogenic and metabolically active state, a faster and more efficient wound healing process can be promoted to improve patient outcomes.

### A. Nanoparticles That Increase Thermogenic Activity in Adipose Tissue

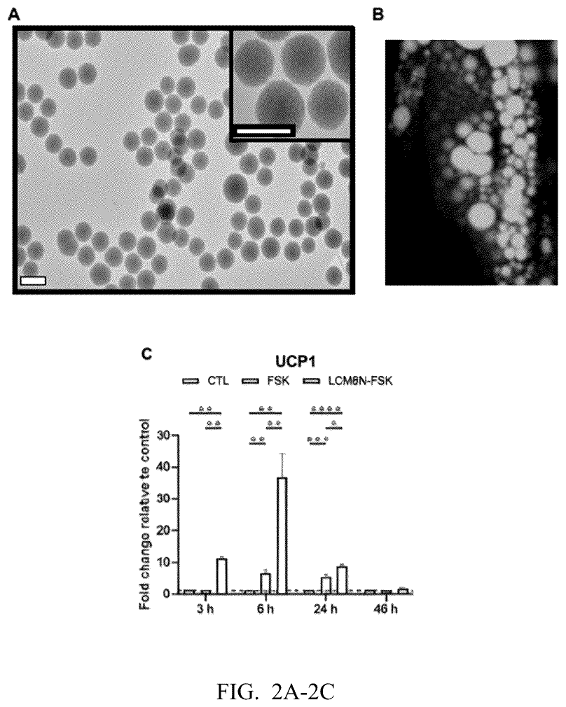

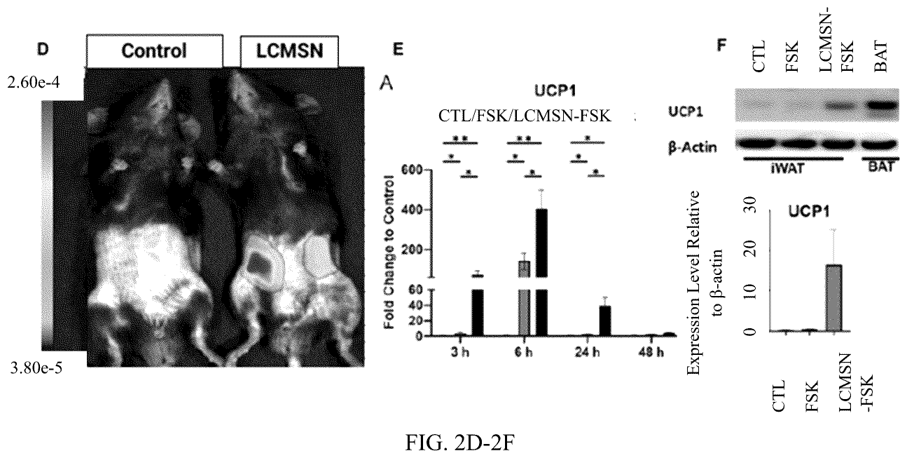

Lipid coated mesoporous silica nanoparticles (LCMSN) technology has been developed for adipose tissue delivery. Mesoporous silica nanoparticles (MSNs) are a type of nanomaterial made of silica (SiO₂) and have a mesoporous structure, meaning they contain pores with diameters between 2 and 50 nanometers. These particles are sized in the nanoscale, typically ranging from 1 to 100 nanometers in diameter. Due to the extensive network of pores, MSNs have a very high surface area which can be beneficial for applications like catalysis, drug delivery, and adsorption. The synthesis methods of MSNs allow for control over pore size, shape, and volume, which can be tailored to specific applications. The silica surface can be easily modified or functionalized with various chemical groups for targeted applications, such as attaching drugs for controlled release or ligands for specific binding. Silica is generally biocompatible, making MSNs suitable for biomedical applications like drug delivery systems or imaging contrast agents. The LCMSN technology consists of fusing liposomal nanoparticles onto premade mesoporous silica nanoparticle (MSN) (FIG. 2A) forming the MSN-supported lipid bilayer, aka LCMSN (31). A LCMSN formulation has been developed that does not interfere with adipocyte differentiation and is taken up by mature adipocytes. Confocal microscopy images showed nanoparticles adjacent to the lipid droplets of human differentiated adipocytes in vitro (FIG. 2B), confirming LCMSN internalization in mature adipocytes. The LCMSN strategy fulfills the three conditions (stable LCMSN, high cargo loading extent, and compatibility to cells).

LCMSN can be loaded with an adenylyl cyclase activator (e.g., forskolin (FSK)) (32-34) that stimulates the thermogenic activity of white adipocytes (7, 14-16). FSK is a bioactive compound whose clinical application has been limited due to the restricted bioavailability (water insoluble) and poor retention in adipose tissue. The nanomaterial-based drug delivery system has been shown to be retained in the sWAT and capable of delivering FSK to sWAT locally. The efficiency of LCMSN loaded with FSK (LCMSN-FSK) to stimulate the expression of thermogenic genes in human adipocytes in vitro was tested. LCMSN-FSK significantly increased UCP1 expression (FIG. 2C). These studies show that LCMSN-FSK can enhance expression of thermogenic genes and metabolic activity and that the effect of adenyl cyclase activator FSK is dramatically enhanced when delivered with LCMSN technology. In biodistribution studies, retention of LCMSN-FSK was observed in the inguinal adipose tissue even after 48 h post-injection when imaging entire animals (FIG. 2D). Importantly, injection of LCMSN-FSK resulted in a significant increase in expression of thermogenic markers including UCP1 (FIG. 2E-2F) compared to free forskolin and controls as early as after 3 h of the injection and until 24 h post injection. These results demonstrate that thermogenic activity can be activated in subcutaneous adipose tissue in vivo as early as 3 h after injection with LCMSNs loaded with forskolin. This tool can be used to understand the effect of increasing thermogenic activity in sWAT during wound healing processes.

### B. Increasing Thermogenic Adipose Tissue Improves Anti-Inflammatory Effects

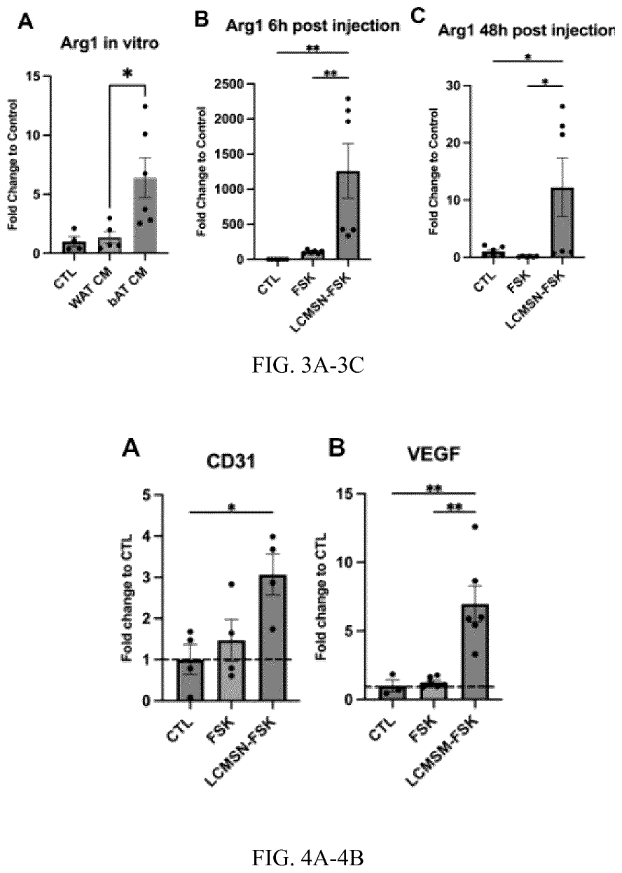

Modulating macrophage activity by inducing the transformation of adipocytes into beige adipocytes could help address dysregulated macrophage activity, which contributes to chronic wounds. For instance, excessive or prolonged M1 macrophage activity hinders proper healing, leading to chronic inflammation and delayed wound resolution. Beige adipocytes can promote the transition of M1 macrophages to the anti-inflammatory M2 phenotype, supporting tissue repair and resolution. Preliminary data demonstrated that macrophages (RAW 264.7) exposed to beige adipocytes conditioned media significantly increase Arginase-1 (Arg1) expression, a hallmark of M2 macrophages (FIG. 3A), compared to macrophages exposed to white adipocytes and control. Importantly, after LCMSN-FSK is injected in the subcutaneous adipose tissue, an increase in Arg1 in mice is observed (FIG. 3B-3C).

### C. Increasing Thermogenic Adipose Tissue Improves Vascularization

Preliminary data demonstrated that subcutaneous injection with LCMSN-FSK significantly increase the gene expression of vascular endothelial growth factor (VEGF) and CD31 in adipose tissue compared to both the control and FSK alone, indicating the potential of our treatment to enhance angiogenesis (FIG. 4A-4B).

### D. Increasing Thermogenic Fat Treats Hypothermia and Frostbite

Cold-related injuries are a significant concern for the military particularly with increasing risk of potential conflicts in cold-weather environments. Frostbite is one of the most common cold-related injuries and requires immediate medical attention. It occurs when tissues are exposed to freezing temperatures for sustained periods of time, leading to local hypothermia and ischemic necrosis. Blood vessel narrowing in the extremities resulting from exposure causes reduced blood flow and oxygen deprivation. Superficial frostbite primarily affects the skin and underlying tissues, while deep frostbite can affect the skin, muscles, tendons, ligaments, and bones. Delayed medical treatment of frostbite can result in gangrene and an increased risk of amputation of the affected body parts and, operational conditions in cold-weather environments may delay access to appropriate hospital care. New techniques that can prevent or treat early stages of frostbite could improve medical outcomes, extend the performance of operational forces, and accelerate return to duty. Additionally, effects of frostbite can be exacerbated in ischemic tissue and tourniquet use may increase the risk of frostbite in extremities due to decreased tissue perfusion, leading to faster cooling of affected limb. Upon the release of the tourniquet, the surge of free radicals during reperfusion phase could also exacerbate the cold-induced injuries in the tissue.

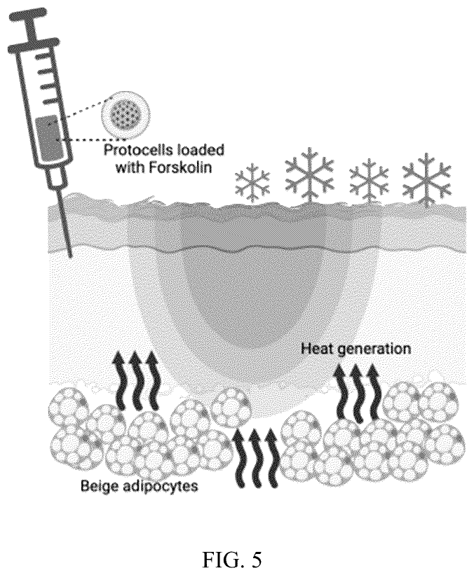

The local stimulation of the thermogenic activity of subcutaneous adipose tissue (sWAT) will prevent, halt and treat tissue experiencing cold burn by increasing local tissue temperature and supporting the wound healing process (FIG. 5). Endothermic organisms regulate their internal temperature by generating warmth through two main processes: shivering and non-shivering thermogenesis. The majority of non-shivering thermogenesis is orchestrated by specific cells in brown adipose tissue. Brown adipose tissues make up less than 1% of the adipose tissue volume in adults and exhibits the capacity for dissipation of energy through increased mitochondrial activity driven, in part, by the expression of uncoupling protein 1 (UCP1) which drives ATP synthesis and stimulates respiratory chain activity. These brown adipocytes generate heat through disruption of the mitochondrial proton gradient through well characterized processes. Thermogenesis primarily occurs in brown adipose tissue; however, brown adipose tissue levels are limited in adult humans and are only able to address moderate systemic cold challenges. In addition, brown adipose tissue is absent in the subcutaneous areas where frostbite injuries typically occur. Significant cold challenges require a larger volume of thermogenic fat in the local tissue environment which may be accomplished by activating thermogenic activity within sWAT. Additionally, inducing sWAT browning has been shown to promote skin wound healing. After injury, sWAT rises to the wound bed during the early stages of healing, helping to close the wound with some adipocytes transforming into fibroblasts to aid in tissue repair. Furthermore, the browning of sWAT regulates immune responses and myofibroblast function. Therefore, increasing the thermogenic activity of sWAT in cold-related injuries not only protects the tissue by raising the local temperature but also enhances wound healing.

Adenylyl cyclase activators, such as forskolin (FSK), have been utilized to stimulate the thermogenic activity of white adipocytes forming so-called beige adipocytes. Forskolin is a bioactive compound whose clinical application has been limited due to the restricted bioavailability (water insoluble) and poor retention in adipose tissue. A nanomaterial-based drug delivery system (described herein) has been developed that is retained in the sWAT and is capable of delivering forskolin to sWAT locally. This technology results from fusing liposomal nanoparticles onto mesoporous silica nanoparticle (MSN) to form MSN-supported lipid bilayer (LCMSN). It has been shown that mice injected with LCMSN-FSK maintained their body temperature better under a cold challenge than those injected with saline. This demonstrates the thermogenic effect of beige fat activation.

### II. Lipid Coated Mesoporous Silica Nanoparticles (LCMSN)

The fusion of liposomal nanoparticles onto mesoporous silica nanoparticle (MSN) forms MSN-supported lipid bilayer or lipid coated mesoporous silica nanoparticles (LCMSN). The term “mesoporous silica nanoparticles” (MSNs) is used to describe nanoparticles according to the present disclosure which are modified to target specific cells in vivo for diagnostic and/or therapeutic purposes. Liposomes or lipid nanoparticles are small, spherical vesicles composed of one or more lipid bilayers or lipids surrounding an aqueous core. Liposomes mimic cell membranes, with a hydrophobic (water-repelling) lipid tail region and a hydrophilic (water-attracting) head region, forming a closed vesicle. Liposomes are used in drug delivery systems because they can encapsulate both hydrophilic drugs in their aqueous core and hydrophobic drugs within their lipid bilayers. This allows for targeted drug delivery, reducing side effects and improving the efficacy of medications. In certain aspects lipid nanoparticles can be used as a delivery vehicle. Lipid Nanoparticles (LNPs) are similar in concept to liposomes but have some distinct characteristics. LNPs are typically composed of ionizable lipids, helper lipids (like cholesterol), PEGylated lipids, and sometimes include other components like phospholipids. They can form various structures like micelles, liposomes, or solid lipid nanoparticles, but are often designed to be more stable and versatile than traditional liposomes. LNPs are particularly noted for their use in nucleic acid delivery, including mRNA vaccines. They protect the nucleic acids from degradation, facilitate cellular uptake, and can be engineered for tissue-specific delivery. Their design allows for controlled release and can be modified for stability, size, and surface properties.

LNPs often have better stability, especially in biological fluids, due to their composition and formulation techniques. While liposomes are great for drug delivery, LNPs are tailored more specifically for nucleic acid delivery, providing protection from enzymatic degradation. Liposomes have a broad range of applications in drug delivery, cosmetics, and food industries, whereas LNPs have seen significant use in gene therapy and vaccine technology. Both technologies leverage the natural properties of lipids to serve as carriers, but they are tailored to different needs in biotechnology and medicine.

LCMSN and MSN of the invention will have an effective average particle size. The phrase “effective average particle size” used herein to describe a multi-particulate (e.g., a porous nanoparticulate or liposomal particulate) means that at least 50% of the particles therein are of a specified size. The LCMSN can be a microparticle (generally defined as a particle with a size ranging from approximately 1 to 1000 micrometers (μm)) or nanoparticle (generally defined as a particle with at least one dimension sized from 1 to 100 nanometers (nm)). Accordingly, “effective average particle size of less than about 2,000 nm in diameter” means that at least 50% of the particles therein are less than about 2000 nm in diameter. In certain embodiments, nanoparticles (e.g., LCMSN) have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm. less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. In certain aspects the LCMSN is larger than 25-50 nm.

In certain aspects a LCMSN composition is administered via intravenous, intramuscular, intraperitoneal, retro-orbital, and/or subcutaneous injection routes. In certain aspect a formulation can be a skin cream (oil-in-water emulsion) incorporating an adenlyl cyclase activator (e.g., forskolin) or a LCMSN described herein for topical administration. The emulsion can be prepared with cetearyl isononanoate, glyceryl stearate, benzyl alcohol, cetearyl alcohol, glycerin, EDTA, and deionized water etc. Dissolvable microneedle array with incorporated encapsulated forskolin is also contemplated. Microneedles are emerging drug delivery methods as rapid, less invasive, and almost painless treatment. Both coated microneedles and dissolvable microneedles can serve drug delivery purposes. Both approaches deliver drugs through the skin in a minimally invasive, painless, and efficient manner, bypassing the challenges of oral or injectable drug administration. Coated microneedles are solid microneedles (typically made from materials like stainless steel, silicon, or polymers) that are coated with a thin layer of drug formulation. The drug is applied to the surface of the microneedles, and when the microneedles penetrate the skin, the drug is released. In certain aspects microneedles are pressed into the skin, penetrating the stratum corneum (the outermost layer of the skin) without reaching deeper nerve endings, making the process painless. Once inserted, the drug coating dissolves or detaches from the microneedles due to the interstitial fluid in the skin. The drug is then absorbed into the systemic circulation or local tissue. After the drug coating dissolves (usually within seconds to minutes), the microneedles can be removed, leaving no residual material in the skin. The microneedles can potentially be reused (after sterilization and recoating), though single-use is more common for safety. Dissolvable microneedles are made from biodegradable or water-soluble materials (e.g., polymers like hyaluronic acid, polyvinyl alcohol, or sugars like trehalose). The drug is encapsulated within the microneedle structure itself, and the microneedles dissolve after insertion into the skin, releasing the drug. Similar to coated microneedles, dissolvable microneedles are pressed into the skin, penetrating the stratum corneum. Once inserted, the microneedles dissolve in the interstitial fluid, releasing the encapsulated drug. The dissolution rate can be controlled by the material composition, allowing for rapid or sustained release. The microneedles fully dissolve, leaving no residual material in the skin. This eliminates the need for removal. Compositions described herein can be incorporated into microneedle delivery vehicles and administered to wounded subjects or subjects having, at risk of having/developing hypothermic injuries.

In certain aspects the administration route can produce long residence times (on the order of at least 12 hours to 2 weeks or more) and/or greater biodistribution and/or bioavailability. The MSN can be monodisperse and generally no greater than about 50 nm in average diameter, often less than about 30 nm in average diameter, as otherwise described herein. The term “D50” refers to the particle size below which 50% of the particles in a multi-particulate fall. Similarly, the term “D90” refers to the particle size below which 90% of the particles in a multi-particulate fall.

The MSN size distribution depends on the application, but is principally monodisperse (e.g., a uniform sized population varying no more than about 5-20% in diameter, as otherwise described herein), but may also be polydisperse in certain instances. The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size.

In certain embodiments, MSNs can range from around 5, 10, 15, 20, 25, 30, 35, 40, 45, 50 nm to around 100, 200, 300, 400, to 500 nm in size, including all integers and ranges there between. The size is measured as the longest axis of the particle. In various embodiments, the particles are from around 5 nm to around 500 nm and from around 10 nm to around 100 nm in size. The mesoporous silica nanoparticles have a porous structure. The pores can be from around 0.5, 1, 2, 3, 4, 5, 6, 7, 8, 9, 10 nm to about 15, 16, 17, 18, 19, 20, 21, 22, 23, 24, to 25 nm in diameter, often about 1 to around 20 nm in diameter, including all integers and ranges there between. In one embodiment, the pores are from around 1 to around 10 nm in diameter. In one embodiment, around 90% of the pores are from around l to around 20 nm in diameter. In another embodiment around 95% of the pores are around 1 to around 20 nm in diameter.

In certain embodiments, MSNs according to the present disclosure are monodisperse and range in size from about 25 nm to about 300 nm; exhibit stability (colloidal stability); haye single cell binding specification to the substantial exclusion of non-targeted cells; are neutral or cationic for specific targeting (e.g., cationic); are optionally modified with agents such as PEI, NMe³⁺, dye, crosslinker, or ligands (ligands provide neutral charge); and optionally, are used in combination with a cargo or therapeutic agent to be delivered to a targeted cell, e.g., sWAT.

In certain aspects the therapeutic agent is an adenylate cyclase activator, including but not limited to forskolin. The adenylate cyclase activator may also be included in LCMSN. Typically the LCMSN can be loaded with cargo or therapeutic agent(s) to a capacity up to about 50 weight % or more (from about 0.01 % to about 50%, about 0.02% to about 40%, about 0.2 to about 35%, about 0.5% to about 25%, about 1 % to about 25%, about 1.5% to about 15%, about 0.1 % to about 10%, about 0.01 % to about 5%): defined as (cargo or therapeutic agent weight/weight of loaded MSN or LCMSN) times 100. The optimal loading of cargo is often about 0.01 to 10% but this depends on the drug or drug combination which is incorporated as cargo into the MSN. This is generally expressed in μM per 10¹⁰ particles where for example values ranging from 2000-100μM per 10¹⁰ particles are included. MSN according to the present disclosure may exhibit release of cargo at pH about 5.5 which is that of the endosome but are stable at physiological pH of 7 or higher (7.4).

The LCMSN can be formulated in a pharmaceutically acceptable solution. The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The surface area of the internal space for loading is the pore volume whose optimal value ranges from about 1.1 to 0.5 cubic centimeters per gram (cc/g). Note that in the MSN according to one embodiment of the present disclosure, the surface area is mainly internal as opposed to the external geometric surface area of the nanoparticle.

The term “lipid” is used to describe the components which are used to form lipid bi- or multilayers on the surface of the nanoparticles which are used in the present disclosure. Various embodiments provide nanostructures which are constructed from nanoparticles which support a lipid bilayer(s). In embodiments according to the present disclosure, the nanostructures may include, for example, a core-shell structure including a porous particle core surrounded by a shell of lipid bilayer(s).

The lipid bi- or multilayer may vary significantly in composition. Ordinarily, any lipid or polymer which may be used in liposomes may also be used in LCMSN according to the present disclosure. Lipids are as otherwise described herein.

Numerous lipids which are used in liposome delivery systems may be used to form the lipid bi- or multilayer on nanoparticles to provide MSN according to the present disclosure. Virtually any lipid which is used to form a liposome may be used in the lipid bi- or multilayer which surrounds the nanoparticles to form MSNPS according to an embodiment of the present disclosure. Lipids for use in the present disclosure include, for example, 1,2-dioleoyl-sn-glycero-3-phosphocholine (DOPC), 1,2-dipalmitoy1-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18: 1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-snglycero-3-phosphoethanolamine (DPPE), 1,2-dioleoyl-sn-glycero-3-phosphoethanolamine-N-[methoxy(polyethyleneglycol)-2000] (18: 1 PEG-2000 PE), 1,2-dipalmitoyl-sn-glycero-3-phosphoethanolamine-N[methoxy(polyethyleneglycol)-2000] (16: 0 PEG-2000 PE), l-Oleoyl-2-[12-[(7-nitro-2-1,3-benzoxadiazol-4-yl)amino]lauroyl]-sn-Glyce-ro-3-Phosphocholine (18: 1-12: 0 NBD PC), l-palmitoyl-2-(12-[(7- nitro-2-l,3-benzoxadiazol-4-yl)amino]lauroyl)-sn-gl-ycero-3-phosphocholine (16: 0-12: 0 NBD PC), cholesterol and mixtures/combinations thereof. Cholesterol, not technically a lipid, but presented as a lipid for purposes of the present disclosure given the fact that cholesterol may be a component of a lipid bilayer according to an embodiment of the disclosure. Often cholesterol is incorporated into lipid bilayers in order to enhance structural integrity of the bilayer. These lipids are all readily available commercially from Avanti Polar Lipids, Inc. (Alabaster, Ala., USA).

MSN of the disclosure may be PEGylated with a variety of polyethylene glycol-containing compositions. PEG molecules can have a variety of lengths and molecular weights and include, but are not limited to, PEG 200, PEG 1000, PEG 1500, PEG 4600, PEG 10,000, PEG-peptide conjugates, or combinations thereof.

Pharmaceutical compositions according to the present disclosure may also comprise an addition bioactive agent or drug. Generally, dosages and routes of administration of the compound are determined according to the size and condition of the subject, according to standard pharmaceutical practices. Dose levels employed can vary widely and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the disclosure include humans, companion animals, laboratory animals, and the like. The disclosure contemplates immediate and/or sustained/controlled release compositions, including compositions which comprise both immediate and sustained release formulations.

In certain formulation embodiments of the disclosure include LCMSN comprised of (MSN) that (a) are loaded with one or more pharmaceutically-active agents and (b) that are encapsulated by and/or support a lipid bilayer. It has unexpectedly been discovered that the administration of LCMSN comprising therapeutic and/or diagnostic agents via intravenous, intramuscular, intraperitoneal, retro-orbital and especially subcutaneous routes of administration at the average diameters indicated above provide enhanced biodistribution, enhanced bioavailability and increased residence time (often at least 12-24 hours to several days up to a week or in certain cases, two weeks to a month or even longer).

Formulations containing the compounds and compositions according to the present disclosure may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages. In certain aspects the formulations can be in preloaded syringes including auto-inject syringes or multiple-inject syringes. The formulations can be incorporated into bandages, gels and other supports that are applied onto or in proximity to a wound or an extremity at risk of hypothermia.

Pharmaceutical compositions according to the present disclosure typically include a conventional pharmaceutical carrier or excipient and may additionally include other medicinal agents, carriers, adjuvants, additives and the like. In some embodiments, the composition is about 0.1 % to about 85%, about 0.5% to about 75% by weight of a compound or therapeutic agent, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion. Liquid compositions can be prepared by dissolving or dispersing the population of LCMSN and/or MSN (about 0.5% to about 20% by weight or more), and optional pharmaceutical adjuvants, in a carrier, such as, for example, aqueous saline, aqueous dextrose, glycerol, or ethanol, to form a solution or suspension.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present disclosure.

The process for making a population of mesoporous silica nanoparticles (MSN) that exhibit a relatively non-uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60,55, 50, 45, 40, 35 or 30 nm (e.g., less than 50 nm, or less than 30, 25, 20, 15 or 10 nm)), a pore size of between about 1 nm to about 200 nm or between about 50, 40, 30, 25, 20, 15, 10, 9, 8, 7,6, 5, 4, 3, 2 or 1 nm, a surface area of between about 100-1,000 m²/g, and optionally a Zeta potential of between about −40 mV to about +40 mV (e.g., greater than 0 mV), the process comprising: (a) preparing a mesoporous silica colloidal solution comprising: a solvent solution comprising: (i) an alkoxysilane selected from tetramethylorthosilicate (TMOS), tetraethylortho silicate (TEOS), tetrakis(2-hydroxyethyl)orthosilicate (THEOS), methyldiethoxysilane (MDES), 3-(glycidoxypropyl)triethoxysilane (GPTMS), 3-(trimethyoxysilyl)propylacrylate (TMSPA), N-(3-triethoxysilylpropyl)pynole (TESPP), vinyltriethyoxysilane (VTES), methacryloxypropyltriethoxysilane (TESPM), diglycerylsilane (DGS), methyltriethoxysilane (MTMOS), trimethylmethoxysilane (TMMS), ethyltriethoxysilane (TEES), n-propyltriethoxysilane (TEPS), nbutyltriethyoxysilane (TEBS), 3-aminopropyltriethoxysilane (APTS), 2-(2,4-dinitrophenylamino)propyltriethoxysilane, mercaptopropy 1 triethoxysilane (TEP MS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), and/or triethoxysilyl-terminated poly(oxypropylene) (ii) a solvent (iii) optionally, a reporter, and a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethylammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., e.g., at about 50° C.; (b) including in said mesoporous silica colloidal solution a composition comprising a primary amine group and, optionally, a PEGsilane compound to produce a nanoparticle containing amine groups; and (c) hydrothermally treating the aminated nanoparticles produced in step (b) by heating the nanoparticles at a temperature of between about 100° C. to about 150° C. (e.g., about between about 110° C. to about 140° C., e.g., between about 115° C. to about 135° C., e.g., at about 120° C.) to yield the optionally PEGylated, monodisperse mesoporous silica nanoparticles (MSN); wherein the process can be one pot or in steps.

In the above process, (a) the alkoxysilane may be 3-aminopropyltriethoxysilane (APTS), the solvent may be N,N-dimethyl formamide (DMF) and the reporter may be rhodamine B isothiocynate (RITC); and the composition comprising a primary amine group is trimethoxysilylpropyl modified polyethyleneimine (50% in isopropanol, M.W. 1500-1800, PEI-silane) and the PEG-silane compound 1s methoxy(polyethyleneoxy)propyl]trimethoxysilane (Mw 550-750, 9-12 EO, PEG-silane).

In an alternative embodiment, a process for making a population of monodisperse mesoporous silica nanoparticles (MSN) that exhibit a relatively uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60, 55, 50, 45, 40, 35 or 30 nm (e.g., less than 50 nm, such as less than 30, 25, 20, 15 or 10 nm)), a pore size of between about 1 nm to about 200 nm or between about 50, 40, 30, 25, 20, 15, 10, 9, 8, 7, 6, 5, 4, 3, 2 or 1 nm, a surface area of between about 100-1,000 m2/g, and a Zeta potential of between about −40 mV to about +40 mV (e.g., less than 0 mV), the process comprising: (a) (1) preparing a mesoporous silica colloidal solution comprising: a solvent solution comprising: (i) an alkoxysilane selected from the group consisting of tetramethylortho silicate (TMOS), tetraethylortho silicate (TEOS). tetrakis(2-hydroxyethyl)orthosilicate (THEOS), methyldiethoxysilane (MDES), 3-(glycidoxypropyl)triethoxysilane (GPTMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycerylsilane (DGS), methyltriethoxysilane (MTMOS). trimethylmethoxysilane (TMMS), ethyltriethoxysilane (TEES), n-propyltriethoxysilane (TEPS), nbutyltriethyoxysilane (TEBS), 3-aminopropyltriethoxysilane (APTS), dini tropheny lamino )propy ltriethoxysilane, mercaptopropy 1 tri ethoxy silane (TEP MS). 2-(3-aminoethy lamina) propy ltriethoxysilane. isocyanatopropyltriethoxysilane, hydroxyl-terminated polydimethylsiloxane, triethoxysilyl-terminated polydimethylsiloxane, methyltriethoxysilane (MTES), and triethoxysilyl-terminated poly(oxypropylene) (ii) a solvent (iii) optionally, a reporter, and (2) a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethylammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., e.g., at about 50°C: (b) including in the mesoporous silica colloidal solution (i) a composition that does not comprise a primary amine group and, optionally, (ii) a PEG-silane compound to produce a nanoparticle containing amine groups (e.g., quaternary, but also secondary and/or tertiary amine) which are not primary amine groups; and (c) hydrothermally treating the nanoparticles produced in step (b) by heating the nanoparticles at a temperature of between about 100° C. to about l 50° C. (e.g., about between about l 10° C. to about 140° C., such as between about l 15° C. to about 135° C., or at about 120°, to yield the optionally PEGylated, monodisperse mesoporous silica nanoparticles (MSNPs); wherein the process can be one pot or in steps.

Formulations of the invention can include 1, 2, 3, 4, 5, or more lipids, including but not limited to DPPC, DSPC, DMPC, DOPE, DOPC, cholesterol, sitosterol, DOTAP, DSTAP, DODMA, DOTMA, DMPG, and/or DSPG.

In additional embodiments, the nanoparticle formulations may comprise alternative encapsulated systems beyond LCMSN, including matrix nanoparticles based on biocompatible polymers or lipids. For example, solid lipid nanoparticles (SLNs) or polymeric matrix particles (e.g., using PLGA or other biocompatible polymers) can be employed, wherein the adenyl cyclase activator (e.g., forskolin) is entrapped within or throughout the solid sphere matrix. Such matrix nanoparticles provide sustained release and improved stability for hydrophobic actives like forskolin. These particles may be prepared by high-pressure homogenization combined with solvent extraction (in-water drying) methods, which allow scalable, reproducible production with controlled particle size and encapsulation efficiency. Liposomal formulations are also contemplated. In such embodiments, a lipid bilayer encapsulates the active agent either in the aqueous core (for hydrophilic partitioning) or partitioned into the intralamellar (hydrophobic) spaces of the bilayer (for hydrophobic actives such as forskolin).

Liposomes can be generated by methods including thin-film rehydration (e.g., hydration of a dried lipid film with aqueous buffer followed by size reduction) and/or high-pressure homogenization to produce uniform unilamellar or multilamellar vesicles. If the active preferentially partitions to the hydrophobic region of the bilayer, micellar formulations may alternatively be used, wherein the active is trapped or enrobed in the hydrophobic core of micelles formed from amphiphilic lipids or surfactants. These alternative formulations provide flexibility in delivery route (e.g., topical, subcutaneous, or transdermal), biodistribution, and release kinetics while maintaining compatibility with the thermogenic activation mechanism described herein.

In the above process, (a) the alkoxysilane is 3-aminopropyltriethoxysilane (APTS), the solvent is N,N-dimethyl formamide (DMF) and the reporter is rhodamine B isothiocynate (RITC); and (b) the composition that does not comprise a primary amine group 1s N-trimethoxysilylpropyl-N, N,N-trimethyl ammonium chloride (50% in methanol, TMAC-silane) and the PEG-silane compound is methoxy(polyethyleneoxy)propyl]trimethoxysilane (Mw 550-750, 9-12 EO, PEG-silane). Additional embodiments are directed to MSNPs and/or populations of MSNPs which are produced by the above methods.

### III. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following examples are designed to determine the efficacy of lipid coated mesoporous silica nanoparticles (LCMSN) as a treatment for frostbite wound healing. (A) Ability of LCMSN to increase thermogenic activity in adipose tissue was determined. (B) Effect of increasing thermogenic activity on inflammation was determined. (C) Effect of increasing thermogenic activity on vascularization was tested. (D) Ability of LCMSN-FSK to treat frostbite was determined.

### A. Nanoparticles That Increase Thermogenic Activity in Adipose Tissue

Results: Lipid coated mesoporous silica nanoparticles (LCMSN) technology has been developed for adipose tissue delivery. The LCMSN technology consists of fusing liposomal nanoparticles onto premade mesoporous silica nanoparticle (MSN) (FIG. 2A) forming the MSN-supported lipid bilayer, aka LCMSN (31). A LCMSN formulation has been developed that does not interfere with adipocyte differentiation and is taken up by mature adipocytes. Confocal microscopy images showed nanoparticles adjacent to the lipid droplets of human differentiated adipocytes in vitro (FIG. 2B), confirming LCMSN internalization in mature adipocytes. The LCMSN strategy fulfills the three conditions (stable LCMSN, high cargo loading extent, and compatibility to cells).

LCMSN can be loaded with an adenylyl cyclase activator (e.g., forskolin (FSK)) (32-34) that stimulates the thermogenic activity of white adipocytes (7, 14-16). FSK is a bioactive compound whose clinical application has been limited due to the restricted bioavailability (water insoluble) and poor retention in adipose tissue. The nanomaterial-based drug delivery system has been shown to be retained in the sWAT and capable of delivering FSK to sWAT locally. The efficiency of LCMSN loaded with FSK (LCMSN-FSK) to stimulate the expression of thermogenic genes in human adipocytes in vitro was tested. LCMSN-FSK significantly increased UCP1 expression (FIG. 2C). These studies show that LCMSN-FSK can enhance expression of thermogenic genes and metabolic activity and that the effect of adenyl cyclase activator FSK is dramatically enhanced when delivered with LCMSN technology. In biodistribution studies, retention of LCMSN-FSK was observed in the inguinal adipose tissue even after 48 h post-injection when imaging entire animals (FIG. 2D). Importantly, injection of LCMSN-FSK resulted in a significant increase in expression of thermogenic markers including UCP1 (FIG. 2E-2F) compared to free forskolin and controls as early as after 3 h of the injection and until 24 h post injection. These results demonstrate that thermogenic activity can be activated in subcutaneous adipose tissue in vivo as early as 3 h after injection with LCMSNs loaded with forskolin. This tool can be used to understand the effect of increasing thermogenic activity in sWAT during wound healing processes.

### B. Increasing Thermogenic Adipose Tissue Improves Anti-inflammatory Effects.

Results: Modulating macrophage activity by inducing the transformation of adipocytes into beige adipocytes could help address dysregulated macrophage activity, which contributes to chronic wounds. For instance, excessive or prolonged M1 macrophage activity hinders proper healing, leading to chronic inflammation and delayed wound resolution. Beige adipocytes can promote the transition of M1 macrophages to the anti-inflammatory M2 phenotype, supporting tissue repair and resolution. Macrophages (RAW 264.7) exposed to beige adipocytes conditioned media significantly increase Arginase-1 (Arg1) expression, a hallmark of M2 macrophages (FIG. 3A), compared to macrophages exposed to white adipocytes and control. Importantly, after LCMSN-FSK is injected in the subcutaneous adipose tissue, an increase in Arg1 in mice is observed (FIG. 3B-3C).

### C. Increasing Thermogenic Adipose Tissue Improves Vascularization

Results: Subcutaneous injection with LCMSN-FSK significantly increase the gene expression of vascular endothelial growth factor (VEGF) and CD31 in adipose tissue compared to both the control and FSK alone, indicating the potential of our treatment to enhance angiogenesis (FIG. 4A-4B).

### D. Increasing Thermogenic Fat Treats Hypothermia

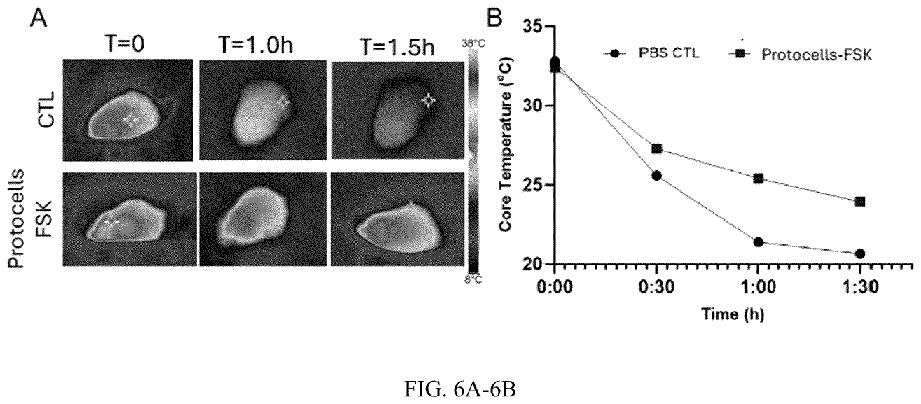

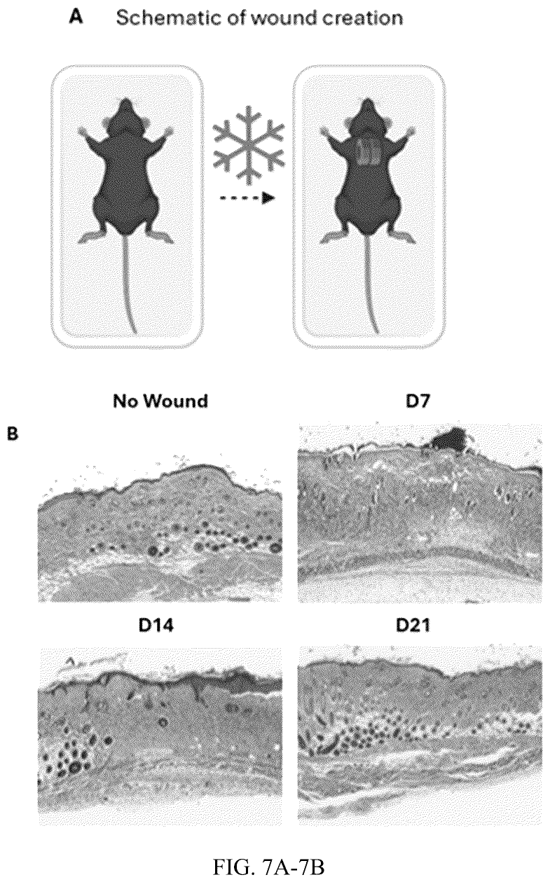

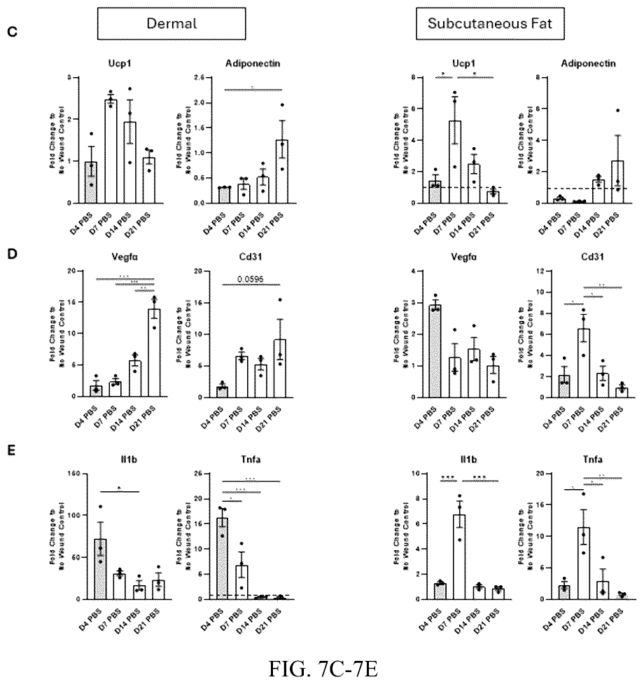

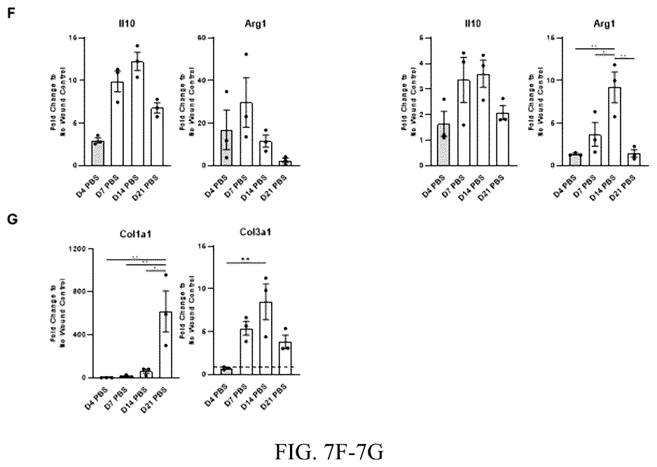

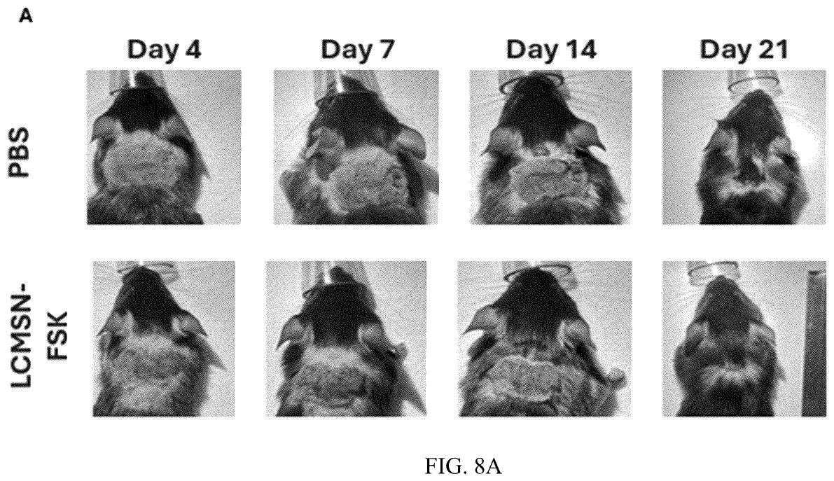

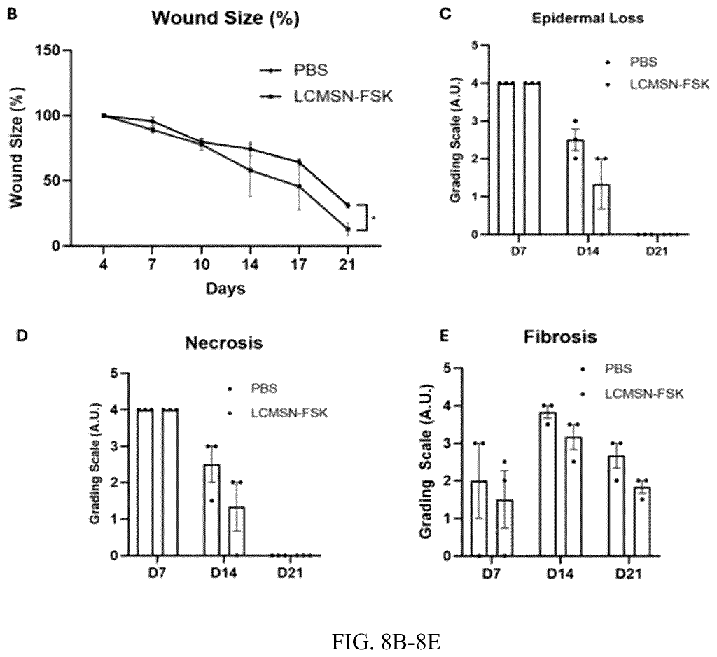

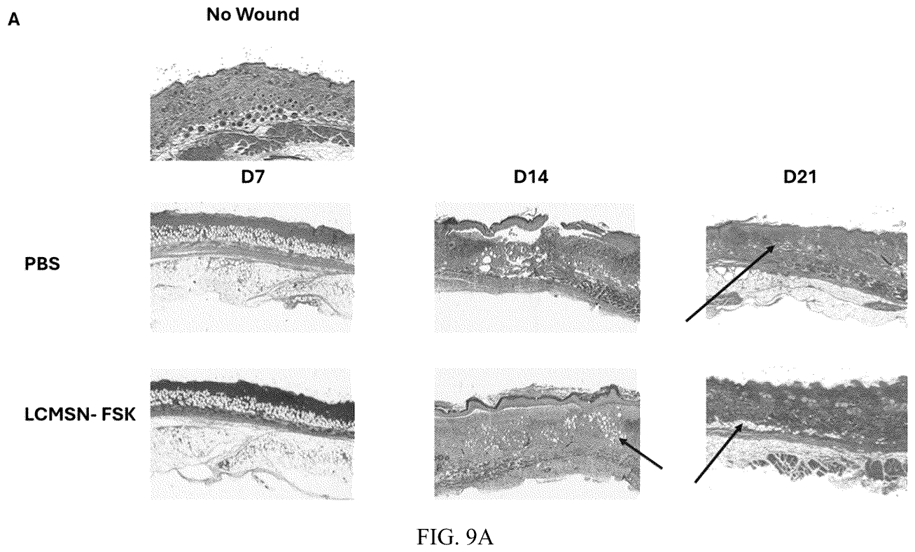

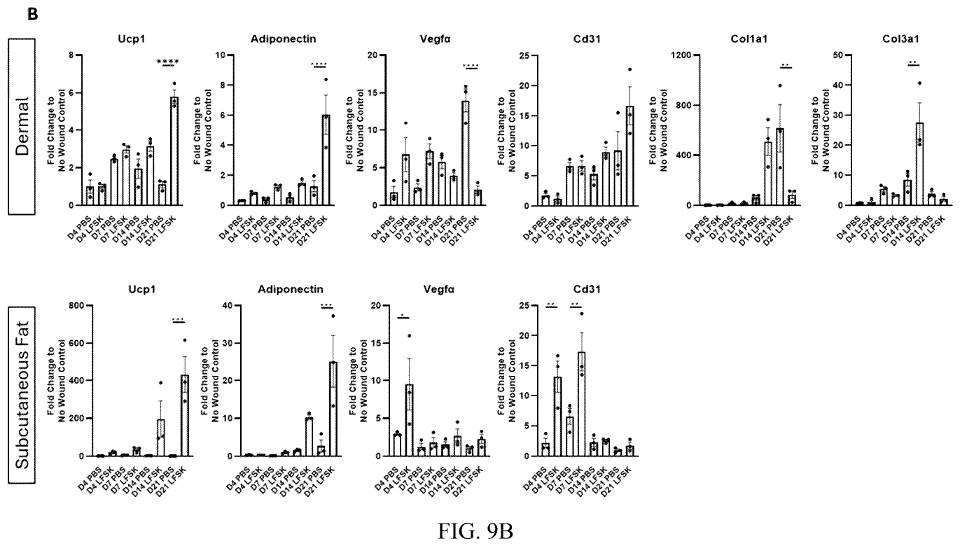

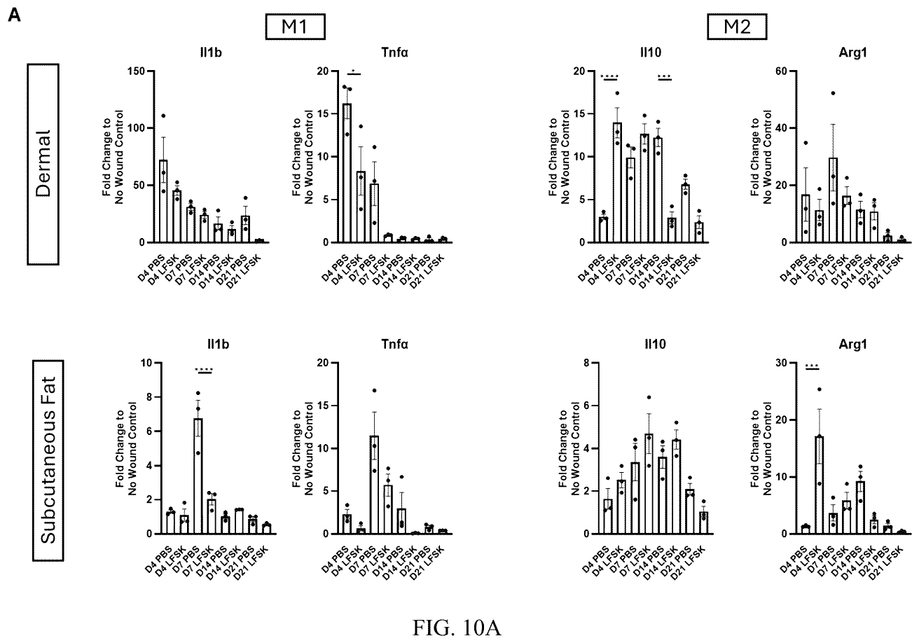

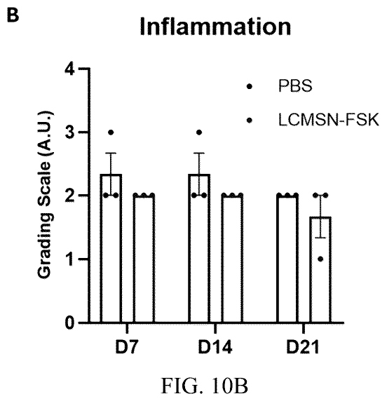

Results: Thermal images of whole mice captured using a FLUKE TiS45 infrared camera of mice undergoing a cold challenge after being injected with either protocells-FSK or saline (CTL) (FIG. 6A). The images show that mice injected with protocells-FSK maintained a higher surface temperature compared to the control (CTL) group. Rectal temperature measurements confirmed that protocells-FSK-treated mice better maintained their core temperature compared to CTL mice (FIG. 6B). These results indicate that protocells-FSK treatment increases core body temperature when mice are challenged with cold.

### IV. Examples

The following examples illustrate embodiments of the invention. The techniques disclosed in these examples represent approaches the inventors have found effective in practicing the invention. Those skilled in the art will appreciate that modifications to these embodiments may be made without departing from the invention's scope.

### Example 1: LCMSN Characterization and Adipocyte Uptake

Lipid-coated mesoporous silica nanoparticles (LCMSN) were fused from liposomes onto dendritic mesoporous silica nanoparticles (pore sizes 4-11 nm). LCMSN showed stability, appropriate size/zeta potential post-forskolin loading, and no cytotoxicity in differentiated adipocytes up to 500μg/mL. Fluorescent LCMSN localized near lipid droplets in human adipocytes (confocal microscopy). Uptake reached 80.6% by 24 h (flow cytometry). LCMSN-FSK significantly increased UCP1 expression in vitro [Zhang et al., 2024].

### Example 2: In Vivo Retention and Thermogenic Activation

Subcutaneous injection of NIR-labeled LCMSN showed retention in inguinal sWAT for ≥48 h (whole-body/ex vivo imaging), with no detectable signal in liver, kidney, heart, or lung at 5 days. LCMSN-FSK increased thermogenic genes (UCP1, Cox7a1) and lipolysis as early as 3 h post-injection, lasting 24 h, outperforming free forskolin. UCP1 protein was elevated by western blot [Zhang et al., 2024].

### Example 3: Temperature Maintenance During Cold Exposure

Mice injected with LCMSN-FSK maintained higher surface (thermal imaging) and core (rectal) temperatures during cold challenge vs. controls, indicating thermogenic protection [preliminary data; extended in frostbite studies].

### Example 4: Frostbite Wound Healing Model and LCMSN-FSK Efficacy

A dorsal frostbite model used-196° C. steel sheet application. H&E showed progressive re-epithelialization over Days 7-21. RT-qPCR revealed temporal changes: upregulated UCP1/Adiponectin (thermogenic), VEGFα/CD31 (angiogenic), reduced pro-inflammatory (Il1b, Tnfα), increased anti-inflammatory (Il10, Arg1), and ECM remodeling (Col1a1, Col3a1) in dermal/subcutaneous layers.

LCMSN-FSK (daily subcutaneous injections post-injury) accelerated wound closure (quantified % reduction vs. Day 4), reduced epidermal loss/necrosis (histopathology scoring), and trended lower fibrosis. Masson's trichrome showed improved ECM remodeling and preserved adipocytes. RT-qPCR confirmed enhanced UCP1/Adiponectin, VEGFα/CD31,Col1a1/Col3a1, reduced Il1b/Tnfα, and increased Il10/Arg1 in treated groups. Inflammation scoring was lower mid-healing [from frostbite manuscript data].

### Example 5: Transdermal Delivery Development

Oil-in-water emulsion creams and dissolvable microneedle arrays were formulated with LCMSN-forskolin. In vitro release/penetration tested via Franz diffusion cells. In vivo application evaluated thermogenic activity in sWAT at 6/24 h (molecular assays). Microneedles showed penetration in pig skin and sustained release (SwRI historical data). Cold tolerance tests (core/skin temperature via infrared camera/implantable capsules) assessed outcomes.

### Example 6: Battlefield-Relevant Model of Tourniquet+Frostbite Injury

A rat hindlimb model was developed combining 2-hour pneumatic tourniquet application with distal frostbite injury (induced by −196° C. contact one hour into tourniquet period) to simulate battlefield conditions of hemorrhage control followed by prolonged cold exposure. Upon tourniquet release, reperfusion occurs in the presence of cold-damaged tissue. Histological analysis 24 hours post-injury revealed muscle fiber disruption, interstitial edema, and hemorrhage, consistent with ischemic and cold-induced damage. LCMSN-forskolin was administered locally (subcutaneously or via emerging transdermal routes) either immediately upon tourniquet application (early intervention by combat medic) or after tourniquet release (at role 2-3 care). Outcomes included limb temperature maintenance (infrared imaging), wound closure kinetics, histopathological scoring of necrosis/edema/inflammation, vascular density (CD31/α-SMA), and molecular markers of thermogenesis (UCP1), angiogenesis (VEGF/CD31), and inflammation resolution (Arg1/IL-10). Preliminary data indicate that early LCMSN-forskolin administration reduces tissue damage severity and accelerates healing compared to vehicle controls, supporting utility in combined ischemic-cold injury scenarios.

### REFERENCES

1. Gonzalez Porras et al., Sci Rep. 2021, 11:5442.
2. Zhang et al., Advanced Functional Materials. 2024.
3. Gurtner et al., Nature. 2008, 453:314-321.
4. Gonzalez Porras et al., Front Bioeng Biotechnol. 2022, 10:906395.
5. Pena and Martin, Nat Rev Mol Cell Biol. 2024, 25:599-616.
6. Franz et al., Dev Cell. 2018, 44:460-470 e463.
7. Shook et al., Cell Stem Cell. 2020, 26:880-895 e886.
8. Correa-Gallegos et al., Nature. 2019, 576:287-292.
9. Cai et al., Cell Metab. 2024, 36:1287-1301 e1287.
10. Gupta, Curr Biol. 2014, 24:R988-993.
11. Horsley, Curr Opin Genet Dev. 2022, 76:101968.
12. Zwick et al., Cell Metab. 2018, 27:68-83.
13. Sakers et al., Cell. 2022, 185:419-446.
14. Li et al., Front Physiol. 2024, 15:1346612.
15. Block et al., Adv Wound Care (New Rochelle). 2015, 4:607-614.
16. Gauglitz et al., Mol Med. 2011, 17:113-125.
17. Pakshir and Hinz, Matrix Biol. 2018, 68-69:81-93.
18. Li et al., Zhonghua Zheng Xing Wai Ke Za Zhi. 2004, 20:124-127.
19. Greaves et al., J Dermatol Sci. 2013, 72:206-217.
20. Mokos et al., Front Med (Lausanne). 2017, 4:83.
21. Coleman, Plast Reconstr Surg. 2006, 118:108S-120S.
22. Sultan et al., Plast Reconstr Surg. 2011, 128:363-372.
23. Coleman, Plast Reconstr Surg. 2002, 110:1731-1744; discussion 1745-1737.
24. Coleman, Clin Plast Surg. 2006, 33:567-577.
25. Strong et al., Plast Reconstr Surg. 2015, 136:897-912.
26. Borrelli et al., Stem Cells. 2020, 38:382-389.
27. Piccolo et al., Clin Plast Surg. 2015, 42:263-283.
28. Guenantin et al., Diabetes. 2017, 66:1470-1478.
29. Ma et al., Front Endocrinol (Lausanne). 2018, 9:473.
30. Fu et al., J Lipid Res. 2005, 46:1369-1379.
31. Durfee et al., ACS Nano. 2016, 10:8325-8345.
32. Wu et al., Cell. 2012, 150:366-376.
33. Scarpace and Matheny, Pflugers Arch. 1996, 431:388-394.
34. Mirbolooki et al., Nucl Med Biol. 2014, 41:10-16.
35. desJardins-Park et al., Adv Wound Care (New Rochelle). 2022, 11:496-510.
36. Hinz, Curr Res Transl Med. 2016, 64:171-177.
37. Hinz, J Biomech. 2010, 43:146-155.
38. Hinz and Lagares, Nat Rev Rheumatol. 2020, 16:11-31.
39. Lampi and Reinhart-King, Sci Transl Med. 2018, 10.
40. Cooper et al., Adv Wound Care (New Rochelle). 2018, 7:299-308.
41. El-Hattab et al., Sci Rep. 2020, 10:10286.
42. Atluri et al., J Orthop Res. 2020, 38:629-638.
43. Villarroya et al., J Endocrinol. 2019, 243:R19-R27.
44. Hondares et al., J Biol Chem. 2011, 286:12983-12990.
45. Li et al., Heliyon. 2024, 10:e30022.
46. Xiong et al., Eur J Pharmacol. 2024, 982:176953.
47. Chen et al., Br J Pharmacol. 2022, 179:1102-1121.
48. Vaicik et al., Endocrinology. 2018, 159:356-367.
49. Eming et al., Science. 2017, 356:1026-1030.
50. Boniakowski et al., J Immunol. 2017, 199:17-24.
51. Krzyszczyk et al., Front Physiol. 2018, 9:419.
52. Buckley et al., Immunity. 2014, 40:315-327.
53. Novak et al., J Leukoc Biol. 2013, 93:875-881.
54. Brancato and Albina, Am J Pathol. 2011, 178:19-25.
55. Yuan et al., Sci Adv. 2024, 10:eadn5229.
56. Jaitin et al., Cell. 2019, 178:686-698 e614.
57. Ntambi and Young-Cheul, J Nutr. 2000, 130:3122S-3126S.
58. Plikus et al., Science. 2017, 355:748-752.
59. Schmidt and Horsley, Development. 2013, 140:1517-1527.
60. Spencer et al., J Clin Endocrinol Metab. 2011, 96:E1990-1998.
61. Cao, J Clin Invest. 2007, 117:2362-2368.
62. Pasarica et al., J Clin Endocrinol Metab. 2009, 94:5155-5162.
63. Engel et al., Biomed J. 2015, 38:58-64.
64. Jiang et al., J Biomed Mater Res B Appl Biomater. 2012, 100:668-676.
65. Jiang et al., Acta Biomater. 2013, 9:4976-4984.
66. Hsiao et al., PLoS One. 2016, 11:e0154328.
67. Moya et al., Biomaterials. 2010, 31:2816-2826.
68. McQuilling et al., Transplant Proc. 2011, 43:3262-3264.

1. A method for treating a wound comprising administering to the wound a nanoparticle comprising an adenyl cyclase activator.

2. The method of claim 1, wherein the nanoparticle is a lipid coated mesoporous silica nanoparticle.

3. The method of claim 1, wherein the adenyl cyclase activator is forskolin.

4. The method of claim 1, wherein administering comprising subcutaneous injection.

5. The method of claim 1, wherein the wound is a chronic wound.

6. The method of claim 1, wherein the wound is frostbite, a diabetic ulcer, a laceration, or a burn.

7. The method of claim 2, wherein the lipid comprises one or more of DPPC, DSPC, DMPC, DOPE, DOPC, cholesterol, sitosterol, DOTAP, DSTAP, DODMA, DOTMA, DMPG, and/or DSPG.

8. The method of claim 1, wherein the nanoparticle is administered topically, via a gel, substrate, or transdermal mechanism.

9. The method of claim 1, wherein administering comprises subcutaneous injection into adipose tissue proximal to the wound or cold-damaged tissue.

10. The method of claim 1, wherein the adenyl cyclase activator increases expression of uncoupling protein 1 (UCP1) in subcutaneous white adipose tissue.

11. The method of claim 1, wherein administration promotes browning of white adipose tissue to beige adipose tissue.

12. The method of claim 1, wherein administration increases thermogenic activity in adipose tissue.

13. The method of claim 1, wherein administration reduces inflammation by promoting transition of M1 macrophages to M2 macrophages.

14. The method of claim 1, wherein administration increases expression of vascular endothelial growth factor (VEGF) or CD31, thereby enhancing angiogenesis.

15. The method of claim 1, wherein the wound is a cold-related injury or frostbite, and administration maintains local tissue temperature or reduces reperfusion injury.

16. The method of claim 1, wherein administration maintains core body temperature during cold exposure or treats/prevents hypothermia.

17. The method of claim 1, wherein the nanoparticle further comprises a second therapeutic agent selected from an antibiotic, anti-inflammatory agent, or growth factor.

18. A pharmaceutical composition comprising a nanoparticle loaded with an adenyl cyclase activator, wherein the nanoparticle is a lipid-coated mesoporous silica nanoparticle.

19. The composition of claim 18, wherein the adenyl cyclase activator is forskolin, and the lipid comprises one or more of DPPC, DSPC, DMPC, DOPE, DOPC, cholesterol, sitosterol, DOTAP, DSTAP, DODMA, DOTMA, DMPG, or DSPG.

20. A method for inducing browning of subcutaneous white adipose tissue comprising administering to the tissue a nanoparticle comprising an adenyl cyclase activator.

21. (canceled)
